(19) United States
(12) Plant Patent Application Publication
(10) Pub. No.: US 2013/0019354 P1
(43) Pub. Date: Jan. 17, 2013

(54) Variety of cherry tree named 'Aramat'

(50) ;

(71) Applicant: Jitka Blazkova, Horice (CZ)

(72) Inventor: Jitka Blazkova, Horice (CZ)

(73) Assignee: Research and Breeding Institute of Pomology Holovousy, Ltd., Horice (CZ)

(21) Appl. No.: 13/135,803

(22) Filed: Jul. 14, 2011

(51) Int. Cl. A01H 5/00

(52) U.S. Cl. PLT/181

(57) ABSTRACT

A new and distinct cherry tree that produces sweet cherry fruits in high quantities.

### BOTANICAL CLASSIFICATION

Prunus avium

### VARIETAL DENOMINATION

‘Aramat’

### BACKGROUND OF THE INVENTION

The present invention comprises a new and distinct cultivar of Prunus avium known by the varietal name ‘Aramat’. The new variety was discovered in Holovousy, the Czech Republic in 1994. The new variety is the result of planned breeding between ‘Krupnoplodnaja’ (Prunus avium ) (female parent, unpatented) and ‘Van’ (Prunus avium ) (male parent, unpatented). The new variety is similar to its parental varieties in fruit skin color, fruit firmness, and flesh color. The new variety differs from its parental varieties in fruit quality, fruit shape, and fruit pedicel length. When compared to unpatented variety ‘Kordia’ (Prunus avium ), the new variety is similar in fruit time of ripening and flesh firmness, but differs in fruit size and shape, as well as susceptibility to cracking. The purpose of breeding the new variety was to provide cherries with improved fruit quality. The new variety has been trial and field tested and has been found to retain its distinctive characteristics and remain true to type through successive propagations. The following characteristics distinguish the new variety from other varieties known to the breeder:

1. Fruit size;

2. Flesh firmness; and

3. Sweet tasting fruits.

### DESCRIPTION OF THE DRAWINGS

The accompanying photographic drawings illustrate the new variety, with the color being as nearly true as is possible with color illustrations of this type:

FIG. 1 is a close-up photograph of the fruit of ‘Aramat’;

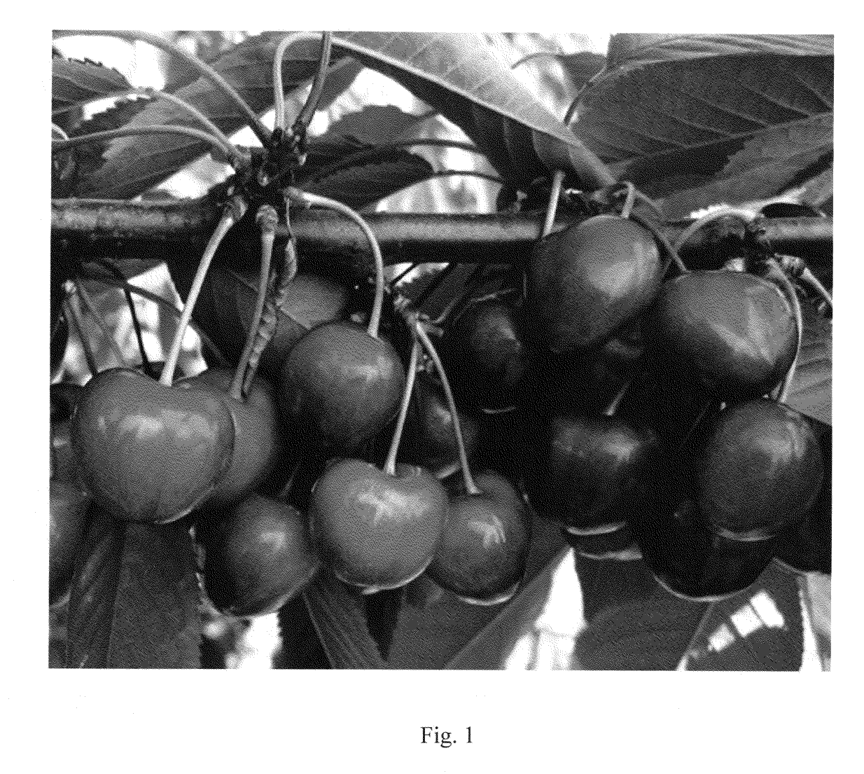

FIG. 2 is a photograph of an entire tree of the new variety; and

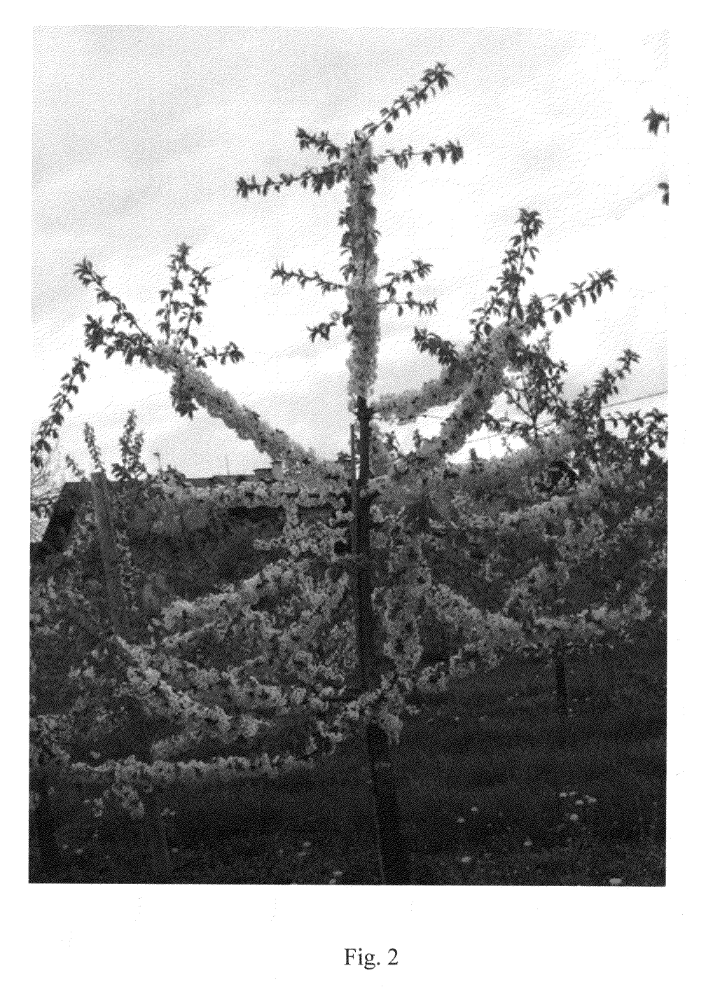

FIG. 3 is a photograph showing measurements of the fruit of ‘Aramat.’

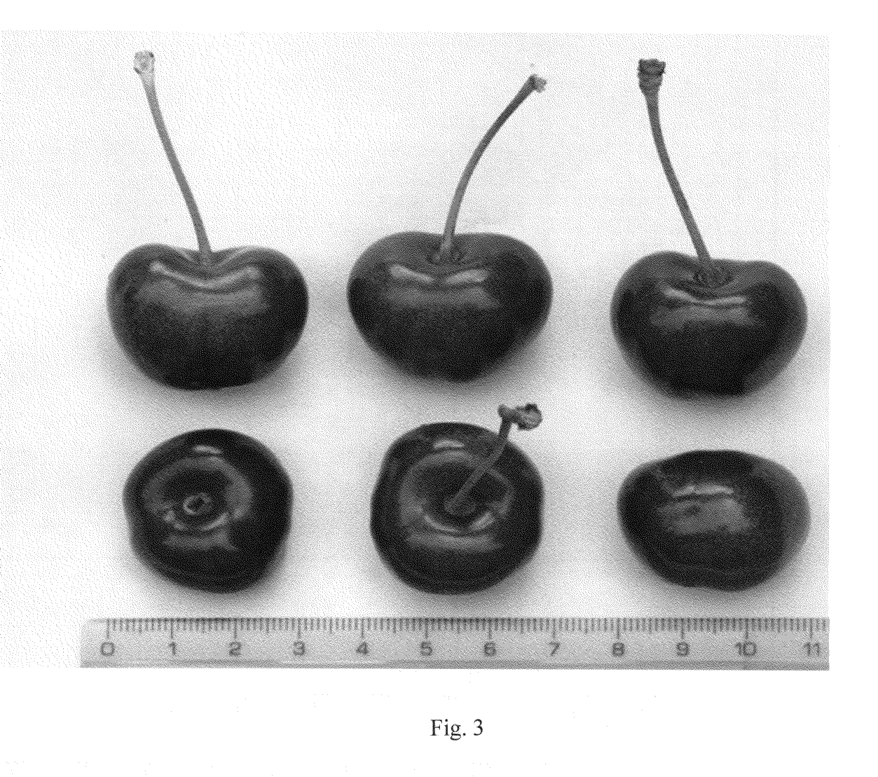

### DETAILED BOTANICAL DESCRIPTION

The following detailed description sets forth the characteristics of the new cultivar. The data which defines these characteristics were collected by asexual reproductions via grafting carried out in Holovousy, the Czech Republic. The new variety was grown in an orchard under standard growing conditions outdoors in the Czech Republic. The color readings were taken in natural daylight, and the plant was 6 years old when described. Color references are primarily to the 1995 R.H.S. Colour Chart of The Royal Horticultural Society of London.
Tree:
   Height.—3.0 m.
   Vigor.—Medium.
   Density.—Medium.
   Form.—Spreading.
   Bearing.—High.
   Growth type.—Standard.
   Rootstock compatibility.—Good with cherry rootstocks ‘Colt’ and ‘Gisela 5’.
Trunk: Diameter: 88 mm. Bark color: 200A. Surface texture: Rough. Lenticels: Length: 10.0-15.0 mm. Width: 1.0-2.0 mm. Color: 175D. Density: High.
Branches: Diameter: At 2 years: 15.8 mm. At 3 years: 17.5 mm. At 4 years: 23.0 mm. At 5 years: 30.0 mm. Surface texture: Smooth. Color: 200B. Form: Slightly drooping. Average angle: 60°. Bud arrangement: 3 to 5 per side. Lenticels: Length: 2.0 mm. on 2 year old trees. Shape: Long and elliptical. Color: 175. Density: Intermediate to high. Shape: Small and rounded early in growth; on older bark, the lenticels are elongated and numerous.
Leaves: Length: 110.5 mm. Width: 52.0 mm. Form: Oval. Texture: Smooth. Thickness: Intermediate. Base: Long and cuneate. Apex: Long and pointed. Margin: Serrate and obtuse. Pubescence: Upper surface: None. Lower surface: None. Color: Upper surface: 135C. Lower surface: 136C. Petiole: Length: 40.0 mm. Diameter: 1.5 mm. Color: Light red color present at the base of the petiole. Vein color: 59B.
Flower buds: Pedicel: Length: 35.0 mm. Diameter: 6.8 mm. Color: 200A. Bud: Length: 15 mm. Width: 3.2 mm. Color: 166A.
Flowers: Blooming timing: Medium early to early. Blooming period: 6-14 days. Number of flowers per raceme: 3-5. Fragrance: None present. Petals: Number: 5. Length: 16 mm. Width: 15 mm. Shape: Oblong. Margin: Entire. Color: Upper surface: White. Lower surface: White. Pollen: Color: Amount (generally): Plentiful.
Fruit (described at full maturity): Size: Diameter: 30.2 mm. Length: 26.2 mm. Skin: Thickness: Medium. Texture: Intermediately fibrous. Tendency to crack: Intermediate. Color: 46A. Flesh: Aroma: Strong. Color: 47D. Texture: Intermediately fibrous. Eating quality: Excellent and sweet. Seeds: Number per fruit: 1. Length: 11.2 mm. Breadth: 10.6 mm. Form: Spherical. Color: 159A. Stem: Length: 40.0-42.0 mm. Width: 1.2 mm.
Tree winter hardiness: Medium.
Bud winter hardiness: Good.
Drought tolerance: Good.

I claim:

1. A new and distinct variety of cherry tree, as is herein illustrated and described.
